(19) United States
(12) Patent Application Publication
(10) Pub. No.: US 2021/0379103 A1
(43) Pub. Date: Dec. 9, 2021

(54) ANISOTROPIC NANOPARTICLE COMPOSITIONS AND METHODS

(71) Applicant: Buckyol Limited, Kowloon (HK)

(72) Inventor: Peter Robert BUTZLOFF, Saint David, ME (US)

(21) Appl. No.: 16/770,434

(22) PCT Filed: Apr. 11, 2018
(86) PCT No.: PCT/US2018/027177

§ 371 Date: Jun. 5, 2020

Related U.S. Application Data
(60) Provisional application No. 62/598,466, filed on Dec. 14, 2017.

(51) Int. Cl. A61K 33/44; A23L 33/10; A61K 47/26; A61K 47/18; A61K 47/12; A61K 47/40; A61K 47/42; A61K 47/46

(52) U.S. Cl. CPC A61K 33/44; A23L 33/10; A61K 47/26; A61K 47/46; A61K 47/12; A61K 47/40; A61K 47/42; A61K 47/183

(57) ABSTRACT

A method for synthesizing a medicinal, nutraceutical, or food fullerene composition, including providing anisotropy in polar and non-polar C60 fullerene hemispheres to create one face of C60 fullerene having a small number of OH-groups clustered to the polar face; providing an amount of a polyhydroxylated fullerene from C60 fullerene; and blending the amount of the polyhydroxylated fullerene with an acceptable ionomer or an acceptable carrier or both. The polyhydroxylated fullerene includes fullerol-'x′ and the amount includes 200 ppm or 500 ppm, wherein ‘x’ is less than 22. The acceptable ionomer includes honey, or a mixture of 3% by wt. sucrose, 1% by wt. proline, 0.2% by wt. magnesium citrate, and 1% by wt. beta-cyclodextrin. The acceptable carrier includes water or a gelatin. A stent, a medical bandage, medical packing material, medical drainage material, acupuncture support, topical ointment, or suture material is impregnated with an anisotropic polyhydroxylated fullerene for antimicrobial action.

### CROSS-REFERENCE TO RELATED APPLICATIONS

This application is related to, and claims benefit and priority under 35 U.S.C. 119(e) to, U.S. Provisional Application 62/598,466, entitled “Antiviral Nanoparticle Ionomer Formulation and Photodynamic Method,” filed on Dec. 14, 2017, which is hereby incorporated by reference in its entirety.

### FILED OF INVENTION

The present invention is related to ionomeric fullerene compositions and, in particular, implantable and consumable ionomeric fullerol compositions, which can be used as a therapy, as a nutraceutical food, or as a nutraceutical beverage.

### BACKGROUND ART

Natural antioxidants such as quercetin from honeybee propolis and bioflavonols in honey have found medical use to treat disease and to confer antimicrobial properties useful in the treatment of wounds. These practices are recorded in ancient texts, and may be at least 5000 years old. Crystalline carbon nanotubes and Buckminster fullerene are regarded as chemically inert materials resistant to oxidation and have been recovered from the black ink writing on ancient papyrus scrolls greater than 3000 years old. However, structural identification and separation of the simplest fullerene from the many compositions and varieties of nanotube was not achieved until recently, for which the 1996 Nobel Prize was awarded. The smallest stable molecule of these carbon forms is considered to be buckminsterfullerene, also known as C60 or [60]fullerene to distinguish it from similar all carbon forms of greater molecular weight. C60 is substantially insoluble in pure polar solvents; however it is slightly soluble in toluene and benzene. Modern pharmacological use of C60 therefore employs derivatives of this molecule that enable them to become soluble in water. Too much functionalization of C60 removes the diffuse resonant characteristic that is spread across the approximately spherical cage structure. This reduces the anti-viral and anti bacterial properties of C60. C60 fullerenes and derivatives are recognized for their benefit of anti-cancer and anti-tumor properties, where such derivatives are largely being commercialized for medical use to help cure a disease such as human immune deficiency vims (HIV), and the Ebola vims, however their long term use and stability raises serious questions that continue to limit their marketability in a preventative or prophylactic mode of treatment.

Pristine or unreacted C60 is recognized as a food additive by the FDA of the United States as an antioxidant. Depending on their degree of functionalization and hydroxylation, water soluble fullerenes may spread to places that are not desirable. In particular, functionalization of C60 greater than about 24 hydroxyl groups may damage mitochondria in cells even if they are much less effective in overcoming the energy barrier of fatty lipids in cell walls for cell entry by cross-membrane transport.

The evolution of resistance to antibiotics with a concurrent rise of human populations, demonstrates that new antimicrobial and antiviral paradigms favorable to humans are needed. Such paradigms will best work within the existing food additive framework, yet act to allow a more targeted method of utility to supplement previously effective methods that are now less and less effectively addressed by the combination of antibiotics and pharmaceuticals used to treat disease. Methods of restricting or mitigating viruses and bacteria in human beings before they become clinical diseases requiring medical intervention are needed. While fullerenes serve as both antioxidants and detoxification agents, and have been marketed as food supplements, the presently sold food products containing fullerenes are restricted to various edible oils, do not easily dissolve or propagate throughout the human body, and are eliminated after a short period of a week or two.

Water insoluble fullerenes temporarily dispersed by lipids have limited solubility at higher concentrations greater than about 30 parts per million, and find no economic applicability in therapeutically significant dosages for human treatment to prophylactic ally avoid disease or illness. Attempts to solubilize fullerenes having no pendant covalent functional groups have met with solution stability problems in the free fatty acids, glycerols, glycerides, vegetable oils, organic esters of fatty acids, phospholipids. In addition, C60 derivative synthesis requires industrial solvents such as toluene or tetrahydrofuran, phase transfer catalysts, or halogen containing intermediates having known carcinogenic or mutagenic properties, and their cleanup for medical grade purity is expensive and time consuming. It is generally recognized that fullerenes have great potential for application as antioxidants and also in photodynamic treatment therapies. If some method of hydroxylation synthesis could be achieved that eliminates exposure to industrial solvents, this would enable compositions of food grade nanoparticles for human consumption.

What is needed is a method and system for effectively managing the dispersion of water insoluble fullerenes, to somewhat increase their solubility and stability in a food or a beverage product, whereby the essential antioxidant and antimicrobial fullerene properties are not compromised by toxic covalent functional chemical attachments or derivatives having unknown long term effects.

### SUMMARY OF THE INVENTION

The present invention provides methods and products using polyhydroxylated C60 fullerene. A method for synthesizing a medicinal, nutraceutical, or food fullerene composition includes providing anisotropy in polar and non-polar C60 fullerene hemispheres to create one face of C60 fullerene having a small number of OH-groups clustered to the polar face; providing a predetermined amount of a polyhydroxylated fullerene from C60 fullerene; and blending the predetermined amount of the polyhydroxylated fullerene with an acceptable ionomer or an acceptable carrier or both. In embodiments, the polyhydroxylated fullerene includes fullerol-‘x’ and the predetermined amount includes about 200 ppm, wherein ‘x’ is less than 22. In other embodiments, the polyhydroxylated fullerene includes fullerol-‘x’ and the predetermined amount includes about 500 ppm, wherein ‘x’ is less than22. In selected embodiments, the acceptable carrier includes about 16 ounces of chocolate. In other selected embodiments, the acceptable ionomer includes a mixture of about 3% by wt. of sucrose, about 1% by wt. of proline, about 0.2% by wt. of magnesium citrate, and about 2% by wt. of beta-cyclodextrin. In still other selected embodiments, the acceptable ionomer includes honey. In still other selected embodiments, the acceptable carrier includes water. In yet other selected embodiments, the acceptable carrier includes a gelatin.

The present invention also provides a medicinal, nutraceutical, or food fullerene composition, including a predetermined amount of polyhydroxylated fullerene; and an acceptable ionomer or an acceptable carrier or both. In some embodiments of the fullerene composition, the polyhydroxylated fullerene includes fullerol-8, and the predetermined amount of fullerol-8 includes about 200 ppm, or 500 ppm. In selected ones of the embodiments of the fullerene composition, the acceptable ionomer includes a mixture of about 3% by wt. of sucrose, about 1% by wt. of proline, about 0.2% by wt. of magnesium citrate, and about 2% by wt. of beta-cyclodextrin. In selected others of the embodiments, the acceptable ionomer includes bee honey. In some embodiments, the acceptable carrier includes water, yet in other embodiments, the acceptable carrier includes a gelatin. In still other embodiments, the acceptable carrier includes about 5% by volume of ethanol. In some selected embodiments, the polyhydroxylated fullerene includes 99.98% fullerol-8. In further embodiments, the polyhydroxylated fullerene includes fullerol-8 stabilized with beta-cyclodextrin and the acceptable carrier includes human blood plasma. In other embodiments, the polyhydroxylated fullerene includes fullerol-8 and wherein the acceptable carrier includes about 16 ounces of chocolate.

Some embodiments include a method of killing or inhibiting the growth of a bacteria or a virus including contacting the bacteria or the vims with an effective antibacterial or antiviral amount of a medicinal, nutraceutical, or food fullerene composition, including a predetermined amount of polyhydroxylated fullerene, and an acceptable ionomer or an acceptable carrier or both. In other embodiments, methods for augmenting the mental acuity of a human, including administering an effective amount of the medicinal, nutraceutical, or food fullerene composition, including a predetermined amount of polyhydroxylated fullerene, and an acceptable ionomer or an acceptable carrier or both. The method embodiment includes administering an effective amount of transcranial direct current stimulation to a human brain. Also provided is a method of treating or managing a toxic chemical ingestion by a mammal, including administering an effective amount of the fullerene composition including a predetermined amount of polyhydroxylated fullerene, and an acceptable ionomer or an acceptable carrier or both. In some embodiments, this method can further include administering a photodynamic therapy.

These and other advantages of the present invention will be further understood and appreciated by those skilled in the art by reference to the following written specifications, claims and appended drawings.

### BRIEF DESCRIPTION OF THE DRAWINGS

Preferred embodiments of the invention will now be described, by way of example, with reference to the accompanying drawings, in which:

FIG. 1 is a view of an ionomer stabilized fullerene formulated in accordance with the teachings of the present invention;

FIG. 2 is a view of beta-cyclodextrin and proline as another ionomer stabilizer formulated in accordance with the teachings of the present invention;

FIG. 3 is a view of a representative fullerene ionomeric network structure in accordance with the teachings of the present invention;

FIG. 4 is a view of a lipid bilayer floating in water-based cell media containing fullerol nanoparticles at the internal non-aqueous lipid-lipid interface, in accordance with the teachings of the present invention;

FIG. 5 is a cross section view of a lipid bilayer containing fullerols that is being breached by an infective virus particle, in accordance with the one embodiment of the present invention;

FIG. 6 is a cross section view of an exploded vims particle and a lipid bilayer containing fullerols that is re-establishing the lipid bilayer after a viral breach, in accordance with the teachings of the present invention;

FIG. 7 is a cross section view of a neuron that is growing a neurite with the assistance of fullerol-8 nanoparticles, in accordance with the teachings of the present invention;

FIG. 8 is a view of transcranial direct current stimulation of the human brain wherein a neuron dosed with fullerol-8 growing a new neurite is shown, in accordance with the teachings of the present invention;

FIG. 9 is a view of a fullerene ionomer formulation during a chemical reaction in accordance with the teachings of the present invention;

FIG. 10 is a block diagram of a method for synthesizing and stabilizing food-grade fullerol, C60(OH)8, from fullerene, C60, without the use of hazardous solvents, in accordance with the teachings of the present invention;

FIG. 11 is a block diagram of a method for synthesizing and stabilizing food-grade fullerol, C60(OH)x, from fullerene, C60, without the use of hazardous solvents, in accordance with the teachings of the present invention;

FIG. 12 is a photodynamic bandage infused with polyhydroxylated fullerenes, in accordance with the teachings of the present invention;

FIG. 13 is a photodynamic salve infused with polyhydroxylated fullerenes, in accordance with the teachings of the present invention; and

FIG. 14 is a view of a medical stent infused with polyhydroxylated fullerenes, in accordance with the teachings of the present invention;

Some embodiments are described in detail with reference to the related drawings. Additional embodiments, features and/or advantages will become apparent from the ensuing description or may be learned by practicing the invention. In the FIGURES, which are not drawn to scale, like numerals refer to like features throughout the description. The following description is not to be taken in a limiting sense, but is made merely for the purpose of describing the general principles of the invention.

### GLOSSARY OF TERMS AND DEFINITIONS

Various additional terms used in the following figures and detailed description are included for providing an understanding of the function, operation, and use of the present invention, and such terms are not intended to limit the embodiments, scope, claims, or use of the present invention.

The term “fullerol-8”, as used herein, is C60(OH)8 unless otherwise specified for the purpose of making a solubility comparison. Fullerol-8, that is octa-hydroxylated fullerene, is composed of C60 bonded with eight hydroxyl groups. Hereinafter, the term “fullerol-8” can represent a single molecular entity or a plurality of such molecules in a dispersed or non-agglomerated nanoparticle state.

The term “fullerol-x”, as used herein, is C60(OH)x unless otherwise specified for the purpose of making a solubility comparison. Fullerol-x, that is x-hydroxylated fullerene, is composed of C60 bonded with x hydroxyl groups. Hereinafter, the term “fullerol-x” can represent a single molecular entity or a plurality of such molecules in a dispersed or non agglomerated nanoparticle state.

The term “photosensitizer,” as used herein, generally means an endogenous catalyst, usually a natural pigment present in the epicuticle such as melanin or eumelanins, or sometimes it is such a pigment produced by a pathological form of microbe. The action of such a catalyst in the presence of light that reacts with such molecules to transfer energy to the tissues of a live animal is to initiate the process of photodegradation and may be consumed in part by such degradation. Photosensitizers play a role in detoxification. Both fullerenes and fullerols may act as photosensitizers in the presence of light energy. These are artificial photosensitizer compounds that are not produced by plants, and are not normally available as food substances produced in nature, as fullerenes are industrially produced at greater than 3000 degrees C. using an electric arc discharge furnace using thousands of volts in an inert gas atmosphere.

The term “singlet oxygen,” as used herein, means a high energy form of diatomic molecular oxygen gas, O2. Its physical properties differ only subtly from those of the more prevalent triplet ground state of O2 gas designated here as 3O2. The terms ‘singlet oxygen’ and ‘triplet oxygen’ refer to the quantum state of the molecules: singlet oxygen exists in the singlet state with a total quantum spin of zero with its electrons remaining in separate degenerate orbitals but no longer with like spin, while triplet oxygen has a total quantum spin of 1 with its electrons having like spin. Singlet oxygen, designated herein as 1O2, is far more chemically reactive toward organic compounds than triplet oxygen. Singlet oxygen can be responsible for the accelerated photo-degradation of many material.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

The present embodiments provide methods and food compositions for human consumption using fullerol-x, effectively managing or limiting many types of infectious microorganisms, and detoxifying the natural exposure of environmental chemicals such as pesticides or other toxic organic poisons capable of impairing function, negatively impacting health, or potentially decreasing normal lifespan by exposure to reactive oxygen species (ROS) such as ozone atmospheric pollutant. Fullerol-8 can combat infective microorganisms and can cleave viral DNA. In addition, fullerol-8 can weaken and destroy tumorous cells, including cancerous cells, and can act to prune away by apoptosis some of the more severely aged or senescent somatic cells. In another related aspect, fullerol-8 can degrade toxic substances accumulated in the human diet, such as a range of chlorinated or unsaturated organic chemicals, especially when activated by light, in which fullerol-8 functions as a photosensitizer in the presence of singlet oxygen. Fullerol-8 can be useful as a radical scavenger and antioxidant to clean up molecular fragment residuals to recover from the effects of singlet oxygen detoxification when light or infrared light is removed and darkness prevails for an extended period.

Dosages of fullerol-8 may be managed by ionomers that may also be formulated without limitation in any type of ionomeric vesicular or ionomeric micellar geometry. Management of therapeutic fullerol dosages also can supply excited states of fullerol by means of near-infrared spectral absorbance ranging from about 700 nanometers to about 1100 nanometers wavelength because of the very large infrared absorbance cross-section.

Barriers to the economic and technological implementation of the fullerenes in medicine include unwanted and undesirable protein denaturing as well as genetic molecular chain scission caused by the excessive water solubility of fullerene derivatives having (—OH) hydroxylation greater than about 12, using an aqueous dosage method. The electron withdrawing and charge distributed resonance effects of fullerol-8 allow significant van-der- Waals polarization of individual molecules, especially when stabilized for long term shelf life using an ionomeric solution. Edible fullerol ionomers can be formulated to especially obtain ionomeric vesicles and micelles as a result of a mechanical shearing operation, wherein the shearing of fullerol-8 in an ionomeric matrix is able to induce the stable retention of as many as 6 electrons of negative charge. Such molecular charge induction allows fullerol-8 to be stabilized for metabolic dispersion and human consumption. The formation of fullerol-8 ionomeric networks are temporary associations based on induced charges and are not chemical points of attachment such as in the formation of a derivative or a covalent bond to a fullerene molecule.

The significant improvement in fullerol dosing of human beings is the development of charge-networked ionomeric hydrogen bonds between organic chemical moieties that act to suspend fullerol-8 into stable agglomerates of about 100 nanometers in a between unlike molecules. This network structure acts to prevent the uncontrolled agglomeration size enlargement and consequent sedimentation and precipitation of fullerol-8 nanoparticles into micron-sized clumps associated with physiological obstruction or toxicological side effects. In the past, such clumps attract and collect dangerous free radicals or attract unusually high concentrations of heavy metal ions in the course of the natural affinity of fullerene for such substances.

Cyclodextrins are enzymatically modified starch molecules typically made by the action upon starch of a natural enzyme termed cyclodextrin glucosyltransferase. Soluble rice starch can be one of the most common feedstocks for this reaction. After cleavage of long linear chains of starch by the natural enzyme, the ends are joined to form a closed circular molecule composed of chemically linked glucose molecules. The three most commonly formed cyclodextrins are alpha, beta, and gamma cyclodextrins, where each of these have six, seven, or eight glucose units present to form the ring structure. Beta-cyclodextrin having seven glucose rings is the cyclodextrin that is produced most commonly by most natural enzymes. The resulting ring of sugars is a very stable polysaccharide having little or no absorption by the digestive system of mammals or insects. Cyclodextrins have the ability to form complexes with a wide variety of organic compounds, which alters their collective solubility, and enhances their stability in the presence of light, heat or oxidizing conditions. Alpha cyclodextrin causes crystallization and precipitation of many amino acids, and therefore is not often used in stability enhancement. Beta-cyclodextrins are significantly used as modern food additives, and both to mask bitterness as well as to protect food flavors from oxidation or precipitation by enhancing their solubility and wrapping the molecule in a protective barrier against reactivity with oxygen species. Beta-cyclodextrins are often used in beverages to prevent the oxidation of fruit juices, and as adjuvants or carriers for pharmaceutical compounds. Gamma-cyclodextrins can be used like beta-cyclodextrins. Use of gamma-cyclodextrins is presently limited due to present market costs of gamma-cyclodextrins.

Cyclodextrins have been extensively used to form covalent bonds with pristine or bare fullerene molecules in attempts to create special pharmaceutical drugs or to increase the solubility of C60 molecules, however there is a strong attraction of fullerene to the inside of such cyclodextrins that hinders their expulsion out of the indigestible cyclodextrin, which acts as a barrier material to prevent fullerene uptake to the digestive system.

Barriers to the application of fullerene-based medicaments and derivatives includes the cost of functionalizing these molecules to have sufficient water solubility to mobilize them for the purpose of administering a therapeutic dosage, yet not use toxic solvents that destroy any long term benefit or therapeutic ability. Therapeutic ability is also reduced by excessive functionalization. Moreover, water soluble fullerene derivatives with greater than 12 (OH) hydroxyl substituents tend not remain sequestered within the bilayer of lipid membranes, as these may significantly migrate to the water-soluble parts of a cell. The present barriers to targeted fullerene dispersion and deployment within specific cell wall lipid interfaces without in-vivo agglomeration have presently been surmounted by the use of the fullerol-8 molecule, which restricts the amount of water solubility, and renders it edible and digestible, as well as stable when dispersed into an ionomeric charge network. Present embodiments provide fullerol-8 ionomers, and methods for employing polyhydroxylated fullerenes, especially that of fullerol-8 molecules, helpful to prevent disease and avoid illnesses.

Neural growth enhancement is enabled by exposure of neurons to static electric charge gradients where the anodic or positively charged region is concentrated at the top of the brain and the upper body surfaces. Nature allows animals the development of positive charges on exposure to air in the presence of wind and charged clouds associated with atmospheric rain storms, as well as the negative grounding effects of the earth and substances in contact with the earth. Recently, the invention of rubber and plastic materials commonly used in footwear has artificially insulated and isolated human beings from natural charge in the environment. Moreover, lifestyle changes associated with moving from an agricultural to an industrialized societies has removed the presence of conductive earth and natural charge gradients from most human environments. Technological human beings who have insulated their feet from earth grounding effects by the use of rubber soled shoes have effectively isolated themselves from therapeutic neural enhancement by natural electrostatic polarization. Some people may elect to submit themselves to short duration, low amperage, low voltage transcranial current stimulation (tDCS) for restoration or enhancement of their natural charge polarity. Even without the presence of fullerol-8, neural growth enhancement and cognitive improvement effects have been documented for more than 30 years in human beings by the now commonly accepted neurological alternative therapy of transcranial direct current stimulation (tDCS). TDCS has been proven to induce irreversible cognitive improvement in human test subjects under a variety of experimental conditions. The tDCS treatment for learning and memory improvement is maximized when a positive charge is applied by an electrode placed at some location near the top of the skull or the upper brain region, whereas the cathode or negative charged electrode tends to provide the best effect when placed at some position lower on the human body, preferentially as low as a wrist or ankle. To some extent, this phenomenon is seen as an alternative way to restore the natural somatic balance of electric charge migration that was removed by the invention of electrically insulating rubber shoes.

Fullerenes and fullerol-8 are highly prone to negative charge accumulation, being able to accept as many as 6 electrons on one molecule. Fullerenes having negative charge tend to migrate through the lipid bilayer of a neural cell to that portion of the neuron where a neurite growth cone is associated with positive charged regions of the cell wall. Filopodia are thin protrusions with a core of parallel bundles of actin filaments that extend from the plasma membrane and explore the cell's surroundings. Filopodia can be found at the leading edge of migrating cells. The growth cone is where the most highly charged regions of the neurite are located at the tips of the filopodia. This effect arises because charges migrate to the tips of pointy objects. Also, actin filaments present their positive charged regions primarily at the tip of the filopodia. The presence of fullerol-8 at filopodia tips can have the effect of destabilizing or weakening the cohesiveness of the lipid bilayer in the cell walls at these locations because of charge balance effects. It is likely that weakened resistance to internal cell turgor pressure supplied by the neuron then causes accelerated filopodia extension and elongated filopodia structures arising from the tip region, yielding filopodia that are suddenly able to reach much further into deep brain structures. The purpose of establishing communication with neurons at a significantly greater distance than would be possible for the network of transmitting and receiving neurons without the assistance of a fullerol-8 neurite growth accelerant, is to enhance whole brain connectivity. The intentional implementation of the chemical and charging effects of fullerol-8 towards the highly strained lipid bilayers of pointed neural structures from within the hydrophobic regions of these cell membranes now provides a surprising and innovative confluence of fullerol-8 antioxidant and anti-microbial protection to sensitive neural structures in confluence with traditional elective tDCS or photodynamic therapies to obtain a synergistic maintenance of human vitality having long term health benefits.

The more deeply networked brain is also the better connected one; the effects of connectivity are fundamental to the educated human being, who must be tasked with learning and managing ever greater amounts of information in modern technological societies. As populations age, susceptibility to disease and neurological deficits increases. Any basis for preventing or reversing this unintentional handicap as a result of increasing life extension will be of significant and immediate economic benefit to the long term vitality of an ever increasing population of human beings. The fullerol-8 dispersion stabilization chemistry by edible ionomeric foods and beverages are anticipated to provide physiological benefit from anti-oxidant and antimicrobial effects as well as to improve the maintenance of long term good neurological health condition of healthy human beings.

Because fullerene and fullerol-8 can be, in general, both a radical scavenger in darkness, and a photosensitizer active with singlet oxygen when exposed to light such as near-infrared light, the presence of fullerol-8 in-vivo confers a combination of therapies having unique functionality different from in-vitro results. Fullerol-8 may migrate to the lipids in the cell wall of blood cells as they would to any cell having a cell wall. Moreover, blood is the medium by which individual fullerol-8 molecules can be brought to the surface of the skin to allow their irradiation even in substantially opaque human beings, where the outer layers of skin are sufficiently translucent to visible light and substantially transparent to infrared light, to allow amplification of traditional photodynamic therapies with oxygenated tissues to produce singlet oxygen. While honey and other foods have a number of natural photosensitizers as a part of their nutritional composition, none of these components can match the light energy harvesting ability of fullerenes, and fullerol-8 in particular. Consequently, human beings subjected to infrared radiation will produce large amounts of singlet oxygen capable of acting in an antimicrobial capacity and especially acting in a detoxification capacity. Human beings are now living longer than ever before, and are therefore able to accumulate but not always eliminate organic toxins over an extended lifetime. Such toxins may include polyaromatic hydrocarbons from smoke, and partly digested food substances. Historically, honey has been used as a human detoxification medicament and antibacterial in the treatment of open wounds to prevent infection and promote healing. These effects in honey are somewhat improved by the presence of natural photosensitizers such as the quercetins obtained from capping sealant on bees wax cells intended to help protect bee brood. However, the addition of fullerol-8 and edible ionomers to beverages and foods will greatly amplify this natural photosensitizer effect with a strong ability by fullerol-8 to generate singlet oxygen on exposure of a wound to air and infrared radiation. While the benefits of red and infrared light in photodynamic therapy have been well characterized and practiced in medicine for decades, in therapies as diverse as skin acne treatment and skin melanoma treatments, it is clear that the addition of an artificial photosensitizer such as fullerol-8 with improved water solubility and enhanced bioavailability will benefit those who elect to spend time outdoors in the natural sunlight, as well as those who elect to undergo the photodynamic treatment method, to be of immediate and complementary practical applicability to better target the defective or infected tissues in many of these well-established therapeutic regimens. Studies have determined that C60(OH)8, with OH-groups forming a compact “island,” constitutes the most bioavailable therapeutic possibility among all the known polyhydroxylated fullerenes. The fairly small number of OH-groups result in a substantially negative hydration energy of −224 kJ per mol. Currently, it is considered a challenge to synthesize such a molecule.

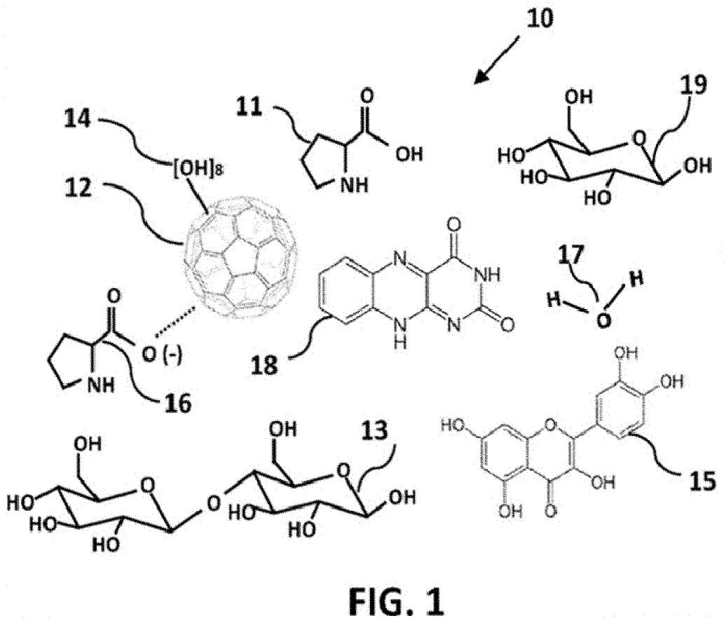

Referring now to the drawings wherein like elements are represented by like numerals throughout, FIG. 1 provides a view of fullerene ionomer 10, where at least one cage-shaped [60]fullerene molecule 12, also known as C60 or buckminsterfullerene, is substantially reacted with eight hydroxyl groups 14 to generate the chemical derivative fullerol-8 or C60(OH)8. The fullerols are Van-der-Waals stabilized for long term storage without precipitation using an ionomeric liquid matrix having a melting point below about 100° C. and composed of ionomers such as the indicated mixture of carboxylic acids including amino acid proline 11, carboxylic anions such as proline anion 16, at least one complex phenolic such as quercetin 15, at least one bioflavonoid such as flavone 18, about 17% of water 17, a multiplicity of saccharides such as glucose 19 and disaccharide sucrose 13. The ionomeric matrix can be composed with honey as one component and may be formulated with supplemental proline anions to enhance the ionomeric stabilization effect on fullerol nanoparticles, wherein the ionomeric formulation is an edible and non-toxic food or medicament serving as a carrier of fullerol-8 (fullerene 12 bonded with eight hydroxyl groups 14), when the ionomeric fullerol mixture dosage is formulated in accordance with an ionomer carrier composition.

One manner in which fullerol-8 may be fabricated is an ultrasound-assisted acoustic cavitation technique, which encompasses a one-step facile reaction strategy, requires less time for the reaction, and reduces the number of solvents used for the separation and purification of C60(OH)8»2H2O. In one such approach, synthesis of water soluble fullerenol (herein, fullerol) via acoustic cavitation can be induced by ultrasound (30% amplitude, 200 W, pulse mode) at ambient temperature, within 1 hour reaction time and in the presence of diluted H2O2 (30%).Adsorption to fullerol-8 is nearly universally stronger than that to C60 fullerenes. While the fullerenes rely solely on van der Waals, π-π, and hydrophobic interactions for adsorption, fullerol-8 nanoparticles also have the capacity for hydrogen bonding while still allowing access to the hydrophobic carbon surface. In a balance of both lipid access and transport with water solubility and transport, the increase in the number of hydroxyl groups from fullerene to fullerol-8 results in increased capability to form hydrogen bonds with water molecules, and thus, increasing water solubility. That fullerol-8 interacted most strongly with most chemicals also suggests that the adsorption of those chemicals is not driven solely by its low water solubility. Therefore, C60(OH)8 (fullerol-8) can provide a potent detoxifying agent for those who ingest it. Ideally, fullerol-8 still needs to be diluted by an ionomer to provide long term resistance to crystallization or precipitation such as in a beverage stored for long periods prior to human consumption.

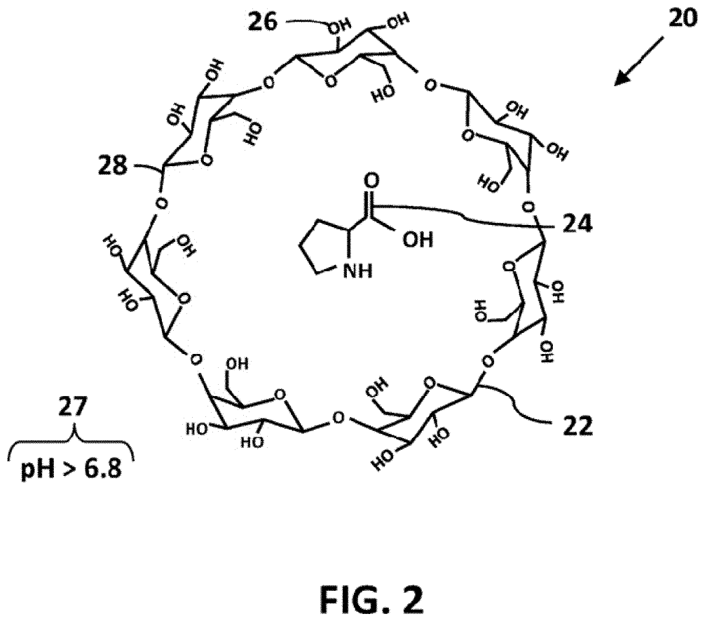

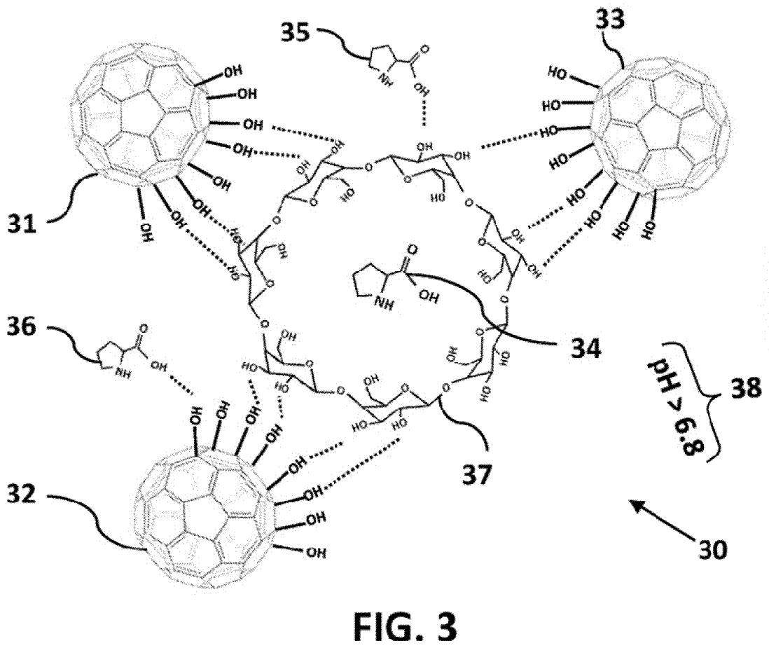

FIG. 2 depicts a molecular host-guest conjugate 20 where the host molecule is beta-cyclodextrin 22 and the guest molecule is proline 24 having an aromatic end containing nitrogen which is non-polar and is slightly attracted to the interior of the cyclodextrin 22, which is also non-polar as indicated by the interior of the beta-cyclodextrin ring 28. The cyclodextrin molecule 22 also has an outward facing side and rim indicated by region 26. Both the non-polar region 28 and the polar region 26 extend around the inside circumference and the outside circumference of the cyclodextrin ring molecule 22. Guest molecule proline 24 will become ejected from beta-cyclodextrin 22 in the presence of an acidic solvent environment having pH less than about 6.8, whereas the guest molecule 24 is stable and remains attracted to the host at the non-polar region when the surrounding pH is greater than a pH of about 6.8 as indicated by 27. Other guest molecules having a non-polar region may be used to dock or associate with the interior of the beta-cyclodextrin molecule, where these may consist of quercetin or flavinol, as shown in FIG. 1. Polar regions of molecules such as glucose, sucrose, or polyhydroxylated fullerene may form hydrogen bonds at the outer circumference or rim of beta-cyclodextrin 26 as will be shown in FIG. 3 below.

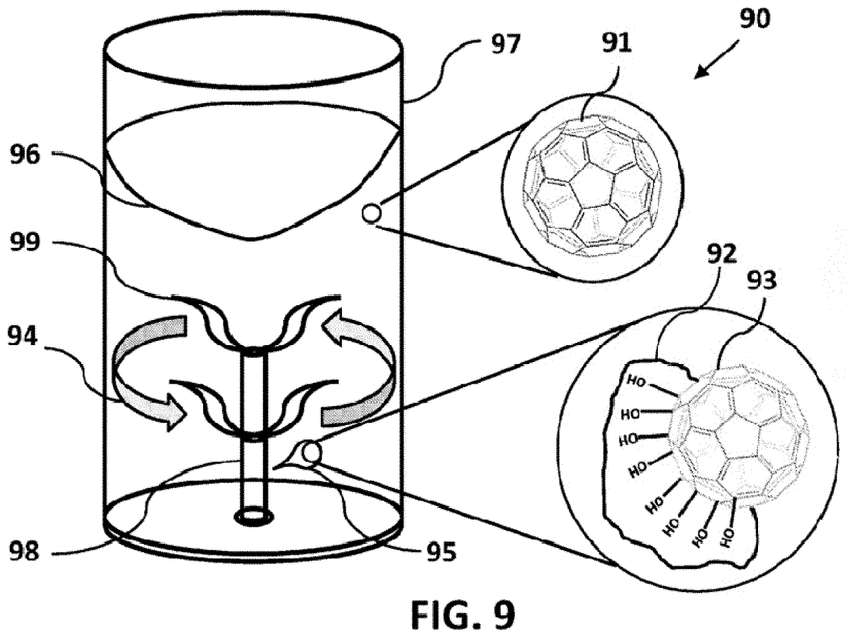

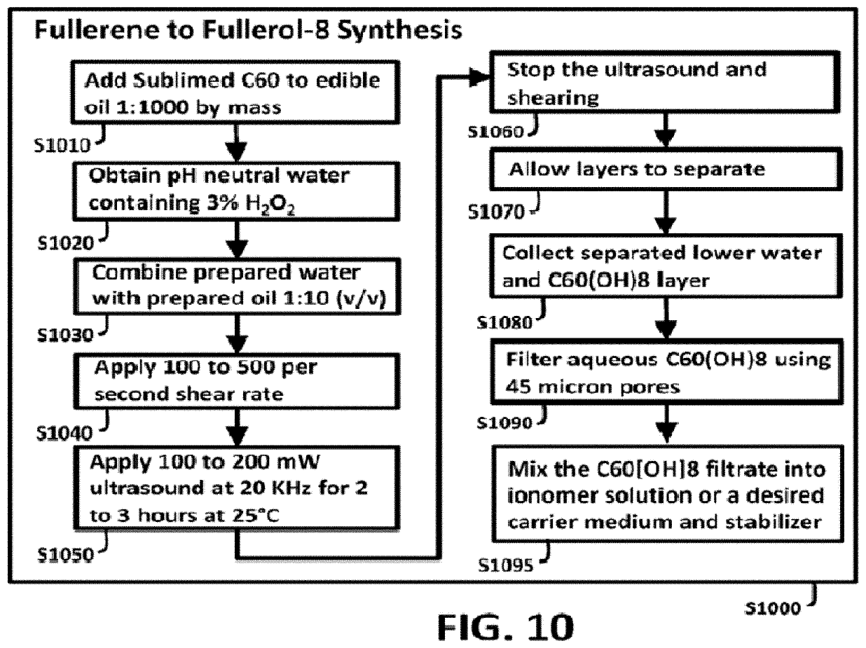

FIG. 3 depicts a network structure 30 among molecules that is more properly termed an ionomer or a self-healing fluid. An ionomer can be tailored to the desired viscosity by appropriate selection of charged, hydrogen bonded, or polarizable molecules capable of forming conjugated associations in liquid media for the purpose of stabilization against sedimentation or precipitation of any of their components as crystals or solid residues. There are no reaction products or polymerizations with, or among, any of the conjugated molecules shown in ionomeric network 30. Solid lines are used to designate the presence of a covalent bond within the molecular structures indicated. Hydrogen bonds in network 30 are indicated by dotted lines used to show these bonds are completely reversible and impermanent associations which do not require the presence of catalysts or a chemical reaction. Hydrogen bonds are capable of randomly reforming in location, position, or orientation on deformation or mixing of the liquid medium, which is most often water. Proline 34 is shown as a conjugated guest molecule in the central ring cavity of beta-cyclodextrin 37. However, another molecule of proline 35 is shown to be hydrogen bonded to the polar hydroxyl functional group at the outside circumference of beta-cyclodextrin 37. Proline 36 is shown to be hydrogen bonded to the polar hydroxyl functional group of fullerol-8 32. Fullerol-8 molecules 31, 32, 33 are shown to be hydrogen bonded to hydroxyl functional groups located at the outside circumference of beta-cyclodextrin 37. The same type of hydrogen bonded structure will form with other cyclodextrins such as alpha-cyclodextrin or gamma-cyclodextrin having 6 or 8 glucose units in their ring structures, respectively. Polymeric versions of the cyclodextrins such as hydroxypropyl-cyclodextrin are equally suitable for hydrogen bonding with anisotropic polyhydroxylated fullerenes in a manner otherwise quite similar to the intent depicted by network 30. Fullerol-8 molecules 31, 32, 33 typically have the formula C60(OH)8, with OH-groups forming a compact island, having a fairly small number of OH-groups highly clustered to one side of the fullerene. This structure forms as the result of one half of the fullerene being exposed to an organic phase which protected it from the hydroxylation reaction, and one half of the fullerene that is highly polar, wherein the details of the synthesis of this structure also are depicted in FIG. 9 and FIG. 10. The pH of the liquid media 38 should be above the pH of about 6.8 to avoid ejection of the guest molecule 34. Proline 34 may be substituted by another molecule capable of forming an incipient conjugation being an impermanent association with beta-cyclodextrin 37. That part of the fullerol-8 molecule not having polar hydroxylations can be incorporated as the guest molecule to be conjugated inside the ring of beta-cyclodextrin, especially when no other potential guest molecules having non-polar regions are present. During the digestion process, acid is present and the low pH of that environment will drop below about 6.7, resulting in the ejection and dissemination of fullerol-8 into the body when the ionomeric composition containing fullerol-8 is eaten.

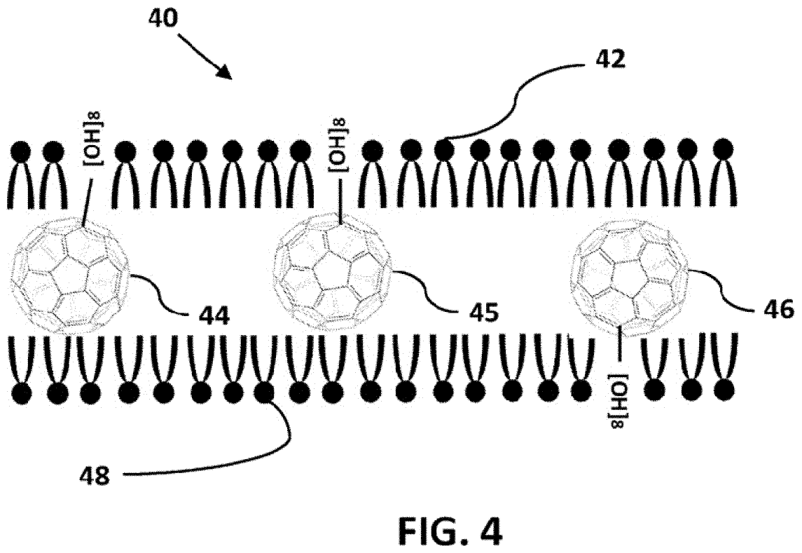

FIG. 4 depicts a cross section illustration of bi-lipid layer 40 having first, “outer” lipid layer 42 with polar molecular head groups facing outward into an aqueous or water based matrix outside a cell, and non- polar tail groups facing inward toward a second “inner” lipid layer 48, having polar molecular head groups facing outward toward the aqueous cell cytoplasm and non-polar head groups facing outer lipid layer 42. Between lipid layers 42 and 48 can be fullerol nanoparticle molecules 44, 45, 46 floating at the internal non-polar interface between the lipid layers 42 and 48 because these molecules find greater solubility and stability at this location than within water based environments. It is depicted that the polar regions of the fullerols 44, 45, 46 tend to face outward into the cytosol or water based media along with the polar head groups in the lipid bilayer, thereby providing enhanced biological oriented interaction of fullerols 44, 45, 56 within stable lipid membranes or liposomes as a result of the anisotropic polar and non-polar fullerol hemispheres. Sequestering fullerols at lipid bilayer 40 can reduce their interference with the aqueous phase DNA and protein machinery of living cells until their oxidative, free radical scavenging, or antimicrobial properties are needed. Fullerols with high numbers of hydroxylated groups, as many as 24 or 32 hydroxylations, may become too soluble in water, and therefore could potentially damage the protein generating machinery of cells on significant exposure to sunlight, because unlike the illustrated C60(OH)8, such highly water soluble (hydrophilic) molecules may not become sequestered at the hydrophobic (water insoluble) internal lipid bilayer of cell walls. In general, solubility of fullerol-x in water increases as x increases, where x is the number of hydroxylations.

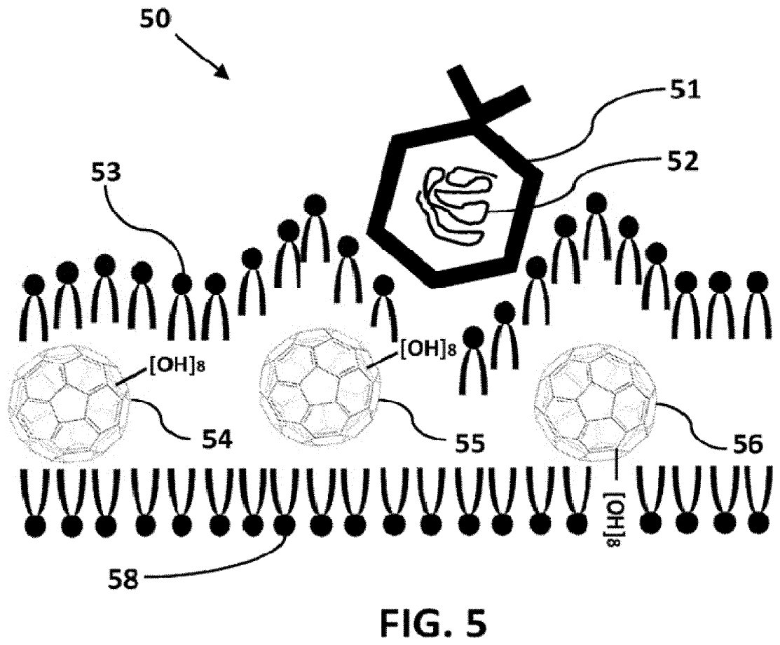

FIG. 5 is a cross-section illustration of a lipid bilayer 50 having lipid layers 53 and 58 containing sequestered fullerol-8 54, 55, 56. The external lipid layer 53 of a cell is being breached by an infective virus particle 51 having a protein coating called a capsid. Typically, capsid 51 is under high internal pressure to contain a tightly packed viral genetic material 52. Capsid 5 1 can be attracted to outer lipid bilayer 53 by a net positive charge that is generally opposing that of the external cell wall, and is also drawn into the cell by the external presence of aliphatic or non-polar functional groups of the capsid that allow access to the inner non polar functional groups of the outer lipid bilayer 53. However, this viral 51 to lipid 53 introduction exposes at least one sequestered fullerol-8 54, 55, 56 of non-polar character that acts to denature the non-polar regions of the viral capsid 51 protein coating sufficiently so that the viral capsid 51 bonding weakens. It is depicted that some of the polar regions of some of the fullerols 54, 55 will tend to face into charged regions of the vims capsid along with the polar head groups in the lipid bilayer, thereby providing enhanced biological and oriented electrostatic interaction of fullerols with an invasive virus as a result of the anisotropic polar and non-polar fullerol hemispheres. The somatic dilution of charge stabilized ionomeric fullerene molecular suspension has extremely strong antiviral properties arising from fullerol-8 opening of viral capsid coatings by exploding the virus particles. These moieties are attracted in dilute form along with some of the charge-stabilized ionomer shell molecules to locations stored within the lipid bilayer of living cells. Virus particles are also attracted to such lipids as a characteristic property enabling viral cellular invasion. Moreover, the fullerol-8 ionomer is able to fit, for example, inside the hydrophobic cavity of HIV proteases, inhibiting the access of substrates to the catalytic site of the viral enzyme.

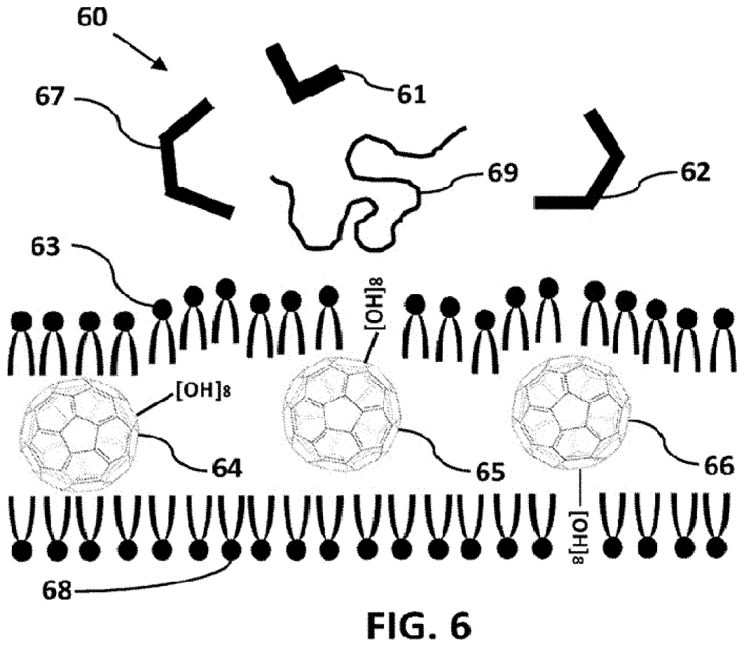

FIG. 6 is a cross-section illustration of lipid bilayer 60 with the molecular remnants of an exploded virus particle showing viral capsid fragments 61, 62, 67 and exposed viral genetic material 69 now residing outside the external cell wall and being susceptible to immune system reaction and expulsion from the intercellular spaces. External facing cellular lipid layers 63 and 68, containing sequestered fullerol-8 as 64, 65, 66, is reforming and becoming contiguous after the viral breach, such that the fullerols 64, 65, 66 have returned to being sealed and sequestered by the cellular lipid layers 63, 68. It is depicted that some of the polar regions of some of the fullerols 64, 65 will take time to re-orient along with the polar head groups in the lipid bilayer, thereby eventually providing stabilized oriented electrostatic interaction of fullerols with the lipids in the cell wall after homeostasis is again achieved, as previously shown in FIG. 4.

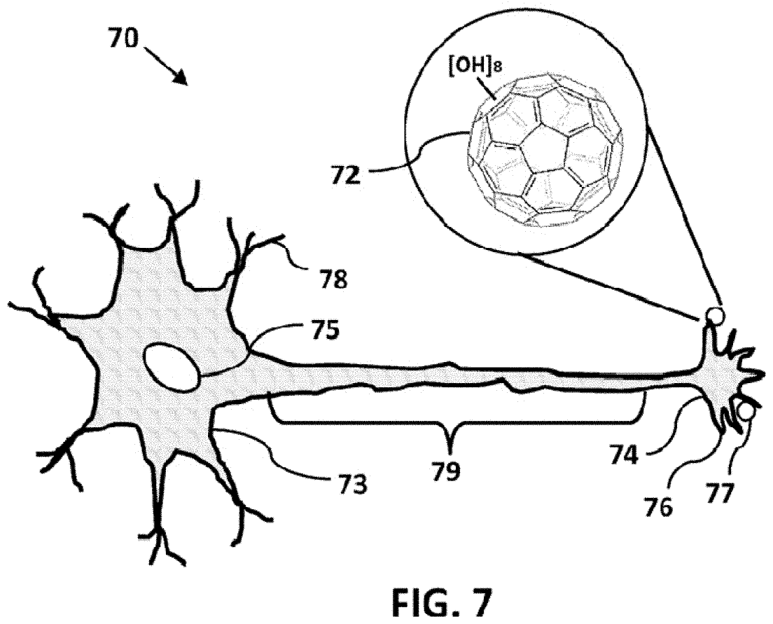

FIG. 7 is a cross section view of treated neuron 70 including mature neuron 73 having single cell nucleus 75, and negatively charged dendrites 78. Neuron 70 functions to receive the input for transmission of signals along the axon conduction path of the neurite 79. Neuron 70 is growing a budded growth cone 74 extending many filopodia 76 with the charge balancing assistance of net negatively charged fullerol nanoparticles 72, 77 being attracted to concentrated positive charged regions of high curvature of the cell wall lipids at the filopodia distal points 76.

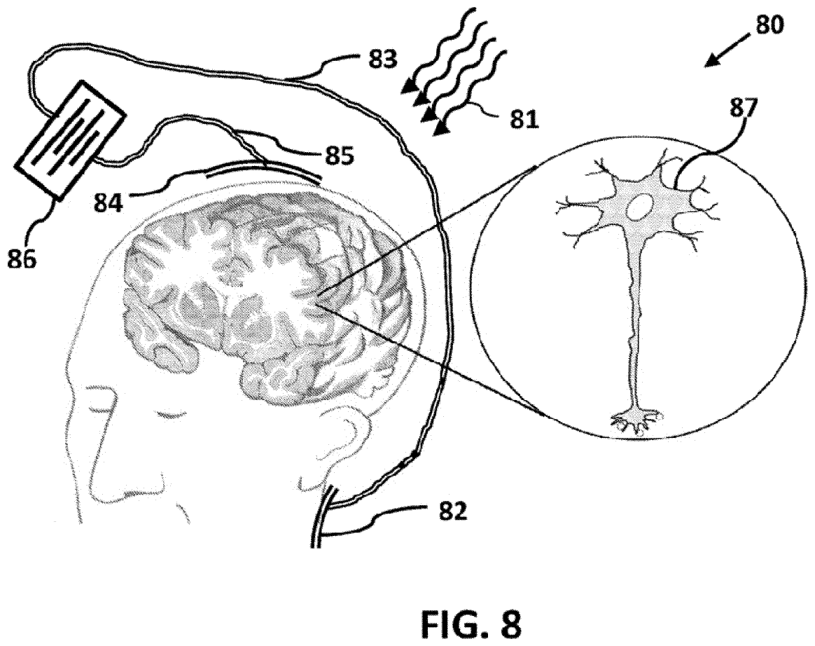

FIG. 8 is a view of a transcranial direct current stimulation (tDCS) device 86 that is providing this well-known therapy to a human being requiring, for example, remedial neurological reconstruction after a stroke, the recovery of neural plasticity in the therapy found to be useful for treatment of post-traumatic stress disorder (PTSD), or for the breakup of beta amyloid plaques associated with Alzheimer's disease. The brain contains a number of different unsaturated fatty acids and has a limited ability to regenerate damaged tissues, making neural tissues very sensitive towards oxidative damage caused by free radicals such as 02⋅-(superoxide), (hydroxyl) and intermediate closed shell H202 molecules. Nano-dispersed fullerenes stop such free radicals from degrading neural fatty acid molecules and membrane proteins, which would lead to glutamate receptor mediated excitotoxicity leading to cell apoptosis (or cell death). Fullerol-8 supports neurite growth in the presence of biogenic electrical current, wherein such neurite growth arises especially from adult oligodendrite cells or adult brain stem cells being encouraged to propagate and grow by the polarization of nano-dispersed fullerenes collected within these cellular membranes. Such neurite growth is achieved by the application of tDCS, thereby assisting with the promotion of electrically induced human neural plasticity, especially in those cases where surgery or pharmaceutical administration by medical intervention is not possible or has not been effective in achieving therapeutic benefit.

The positive or anodic tDCS electrode 84 is shown providing positive charge by conduction wire 85 to the top of the of the human head and by transmission through the tissues and cranial bones, to the underlying tissues of the brain wherein a neuron dosed with fullerol-8 growing a new neurite is shown in an expanded view 80 for context in a brain cross section view. Negative charge is provided at some arbitrary lower point in the body, herein shown to be the neck region, at the location of cathodic electrode 82 obtaining current from conductive wire 83. Wires 83, 85 connect to the transcranial direct current stimulator 86 which may generate power by a battery source or by transformer in connection to a conventional house current AC supply that is capable of performing electrical rectification and smoothing for the production of DC electricity. Both insoluble fullerenes and soluble fullerene derivatives are capable of dissolving beta amyloid plaques by destabilizing charged regions of sharp protein curvature that are organized in arrays within these organic crystal plaques. Moreover, these plaques tend to collect at neurite filopodia used to make connections to dendritic structures of neurons. The application of electric charge induces the migration of fullerenes to the filopodia tips as well as sharp points in the beta amyloid crystal structures where the regions of arrayed high curvature can be most easily disrupted. Thus, the therapeutic combination of charge orienting fullerenes by electricity as well as the chemical denaturing process of beta amyloid protein plaques by fullerenes serve a synergistic and complementary targeting role in the illustrated method of avoiding neurological disease in FIG. 8. The extension of neurites by fullerenes to grow past regions of obstruction can be achieved by this treatment using both fullerene and tDCS. While the denaturing of undesirable protein also may be advantageous in regard to this method, it is important to disrupt and destroy the denatured protein causing neurological impairment. Thus, one use of fullerene may be to eliminate the beta amyloid plaque from the extracellular regions in the brain. Herein, the photodynamic therapy may be subsequently or simultaneously applied to the human patient as shown by infrared irradiance 81 which has the purpose of inducing fullerenes carried by the blood to become excited, and these excited states are thereby transferred to ordinary dissolved oxygen molecules to create reactive singlet oxygen molecules. At this point, the combination of catalytic excited fullerene and singlet oxygen serve to destroy the denatured imperfect proteins responsible for neurological impairment.

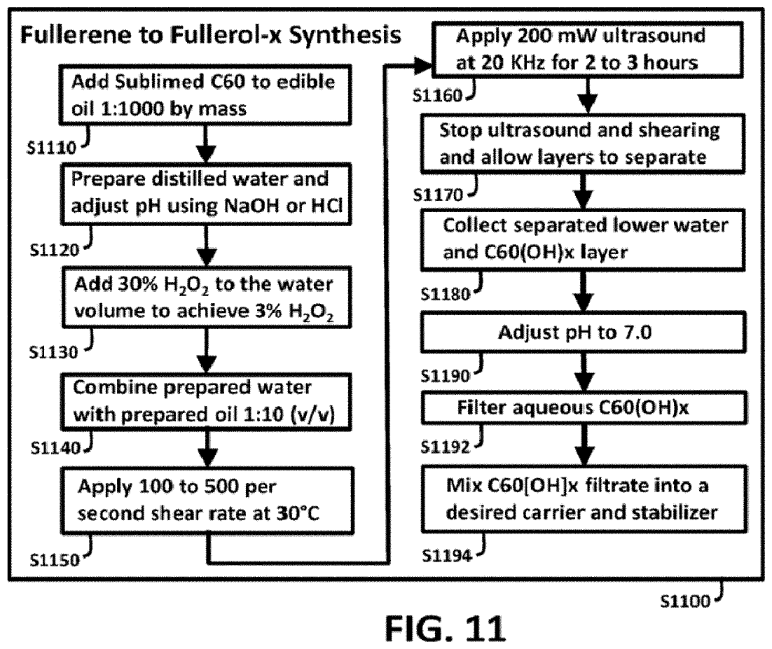

FIG. 9 is a view of a fullerol-8 synthesis 90, where fullerene reactant 9 1 is converted to fullerol-8 product 93 at mixing chamber 97. The liquid medium is composed with a polar phase solvent such as desirably water, and a non-polar phase liquid such as desirably an edible oil. The presence of hydrogen peroxide, H202, in the polar water phase removes non-fullerene carbon. The impurity amorphous carbon can be present even when the reactant raw material, pristine or unreacted fullerene, has been prepared in a benzene solvent split-flame process to maximize purity, even after a vacuum sublimation purification process. The polar and non-polar mixture is filled to the level of the meniscus fill line indicated by the surface of the liquid to air interface 96. Multiple mechanical shearing vanes 99 are caused to spin about impeller shaft 98 in at least one direction of mechanical spin maintained as indicated by the two large arrows 94 to show the direction of continuous rotational movement of the shearing vanes. The temperature of this process is above the freezing point, and below the boiling point, of the liquid mixture components, and can be about 25 degrees C. Micellar formation having a bubble-like structure as small as about 5 to about 10 microns can be formed in this process because of the presence of dispersed polar phase droplets in this mixture, which is desirably distilled water. Shearing rates of at least about 10 per second, and preferably of about 100 to about 1000 per second, achieve the frictional molecular charging objective for temporary nanoparticle stabilization against sedimentation of the fullerene reactants 91 as a precipitate before long term stabilizers are added. The application of ultrasound of about 200 milliwatts and about 20 kilohertz is used for the polyhydroxylation reaction. Ultrasound acts to disperse and activate the crystalline pristine fullerene raw material. The presence of both water, as a polar phase, and edible oil, as a non-polar phase, causes the fullerenes to have one half of their cage in either liquid medium during this process. Fullerol 93 can be formed in this manner, to generate OH-groups forming a compact island, having a fairly small number of OH-groups highly clustered to one side of the fullerene. This structure can form as the result of one-half of the fullerene being exposed to an organic phase, which protects it from the hydroxylation reaction, and one half of the fullerene that is exposed to an highly polar water solvent, which is substantially immiscible in the non-polar solvent. This results in an anisotropic reaction product that constitutes a geometric feature of the class of polyhydroxylated fullerenes having low numbers of hydroxylations. Shearing causes the micellar regions to form characteristic teardrop shapes 95, and to collect fullerol nanoparticles having a wake or cometary geometry, as depicted by skirted region 92. Skirted region 92 includes the water containing polar phase at the tailing end of the moving polyhydroxylated fullerene 93, where the nucleation of even one hydroxylation can be sufficient to create an anisotropic region of polarity on the fullerene. Skirted region 92 is attractive to water and polar reactive oxygen species, which attach preferentially to one side of the spherical shape of the fullerene while it is in hydrodynamic motion. Such vesicles 95 can be shown in context by one such teardrop shaped water vesicle 95. This state of multiple vesicles is formed in a mixture of polar and non-polar immiscible solvents under dynamic shearing conditions where there is a flow and a gradient of frictional molecular forces that act differently for each type of solvent across the fullerene surface. This anisotropy in polar and non-polar solvent faces creates one face of fullerene 93 having a small number of OH-groups highly clustered to the polar solvent face, while no such groups are at the non-polar face of the fullerene in the non-polar solvent. The different friction or attractiveness of the particle in either fluid in a shearing field is a type of dynamic differential nanotribology. Hydrogen peroxide is desirably present in the water phase 92, as a reactant which is used to help accelerate the conversion of fullerene to polyhydroxylated fullerenes, as described in the synthesis process of FIG. 10 and FIG. 11, below.

FIG. 10 illustrates the method developed herein to disperse fullerene in oil and synthesize soluble fullerol-8, with eight hydroxyl groups, using USP food grade edible components in each operation of S1000. To begin in step S1010, the vacuum sublimed grade of fullerene C60 can be obtained from a commercial vendor or certified to assure no trace of toxic solvents, used to purify fullerene, are left in the product to ensure it is a food grade C60. Weigh about 1/1000 by mass of this black crystalline material to a mass of food grade edible oil such as corn oil or sunflower oil. In step S1020, obtain or prepare USP food grade 3% by volume of USP food grade hydrogen peroxide in water. In step 1030, combine the prepared peroxide water with the prepared fullerene in oil in a volume ratio of about 1:10 respectively. In step S1040, apply about 100 to 500 per second shearing rate to the combined mixtures at room temperature or 25 degrees C. In step 1050, apply 100 to 200 milliwatts of ultrasound at 20 kilohertz for about 2 to 3 hours to assure the dispersion and reaction of fullerene. This point can be observed visually when the black solid fractions of suspended fullerene crystals have reacted to create liquid white fluid of dispersed fullerol-8 in water. Fullerols tend to accumulate in vesicles that swirl near the bottom of the reaction vessel. In step S1060, stop the shearing and ultrasonic processes. In step S1070, allow the upper oil layer to separate completely from the lower water layer which is now colored white. In step S1080, collect the fullerol-8 layer for filtration. In step S1090, collect the white colored lower layer containing water and fullerol C60(OH)8 product, and filter this solution through a filter having no greater than about 45 microns of pore size to ensure the substantial removal of any non-dispersed materials. In step S1095, mix the artificial antioxidant and light activated photosensitizer C60(OH)8 solution into the carrier medium such as an acceptable and suitable ionomer to create a long term stable master batch dispersion, as illustrated by the molecules of FIG. 1, FIG. 2, and FIG. 3. It should be noted that this method to synthesize fullerene-8 produces food grade or medical grade polyhydroxylated fullerenes, and does not use any phase transfer catalyst or industrial solvents, since such substances are poisonous, even in trace quantities. Together, FIG. 9 and FIG. 10 depict use of edible oil as a reaction medium to synthesize a food grade polyhydroxylated fullerene.

FIG. 11 illustrates the method developed herein to solubilize and synthesize fullerol-x with variable number of hydroxyl groups. These fullerols are made from fullerene using USP food grade edible components in each operation of S1 100. To begin in step S1110, the vacuum sublimed grade of fullerene C60 is obtained from a commercial vendor to assure no trace of toxic solvents used to purify fullerene are left in the product to ensure it is a food grade C60. Weigh about 1/1000 by mass of this black crystalline material to a mass of food grade edible oil such as corn oil or sunflower oil. In step S1120, prepare the desired pH of a volume of distilled water. The pH should be greater than about 3.0 to avoid the formation of carboxylic acid on the fullerene or this method fails to produce fullerol-x. Select the increasing pH to obtain increasing amounts of hydroxylations (x). At pH greater than about 7.0, the addition of sodium hydroxide will ultimately generate hydroxyl groups by temporarily displacing hydrogen with sodium, to form the functional group (—ONa). More such groups will be added as the target pH is increased above pH of about 7. Note that adjusting pH to approximately pH=7.8 produces mostly fullerol-9, and approximately pH=12 produces mostly fullerol-30, whereas approximately pH=6 produces mostly fullerol 7. In step S1130, add a sufficient amount of USP grade 30% hydrogen peroxide to prepare 3% by volume of pH-adjusted hydrogen peroxide in water. In step S1 140, combine the prepared pH-adjusted peroxide water with the prepared fullerene in oil using a volume ratio of about 1:10 respectively. In step S1150, apply about 100 to 500 per second shearing rate to the combined mixtures at room temperature or 25 degrees C. In step S1160, apply 100 to 200 milliwatts of ultrasound at 20 kilohertz for about 2 to 3 hours to assure the dispersion and reaction of fullerene. This point can be observed visually when the black solid fractions of suspended fullerene crystals have reacted to create liquid white fluid of dispersed fullerol or sodium fullerol nanoparticles in water. Fullerols tend to accumulate in vesicles that swirl near the bottom of the reaction vessel. In step S1170, stop the shearing and ultrasonic processes. In step S1180, collect the separated water layer containing fullerol or sodium fullerol layer which is now colored white. In step S1 190, adjust the pH of the water solution with HCl (hydrochloric acid) or NaOH (sodium hydroxide) as needed to achieve a neutral pH at Ph. In step S1192, filter the pH neutral fullerol solution through a filter having no greater than about 45 microns of pore size to ensure the substantial removal of any non-dispersed or insoluble materials. In step S1195, mix the artificial antioxidant and light activated photosensitizer C60(OH)x solution into the carrier medium such as a desired ionomer to create a long term stable master batch dispersion., as illustrated by the molecules of FIG. 1, FIG. 2, and FIG. 3.

This synthesis method produces a food grade additive useful at trace concentrations that is able to maintain human health by strong antioxidant properties of hydroxylated and polyhydroxylated fullerenes (fullerol-x), especially that of the preferred fullerol-8, and to reduce or eliminate the presence of toxic accumulated biological residues accumulated over a lifetime by means of light-mediated degradation and detoxification by the fullerol-8 photosensitizer component through natural sunlight or photodynamic treatment, and can also be used to improve the treatment of transcranial direct current stimulation, when used in accordance with the intent of the present invention.

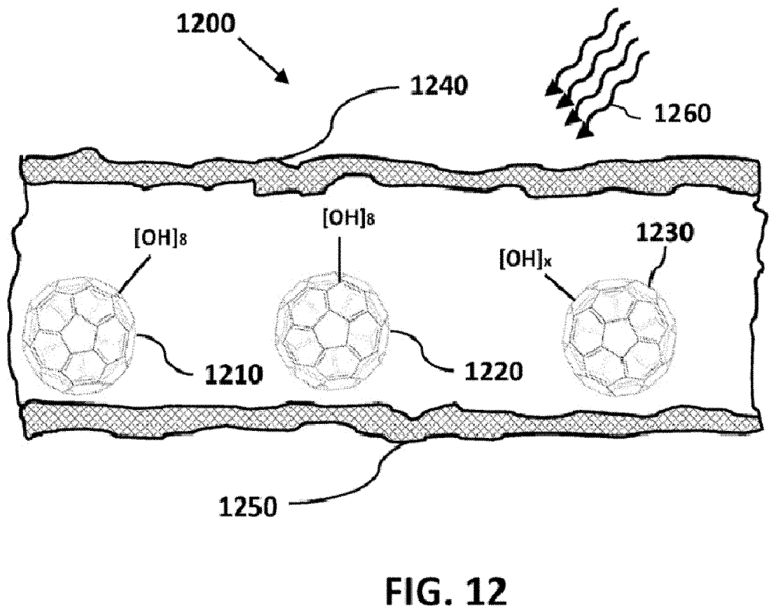

FIG. 12 depicts bandage or suture 1200 having exterior exposed surfaces 1240, 1250. Bandage or suture 1200 carrier material is often made with chitosan and may also have components such as cross-linked hyaluronic acid or processed cartilage as part of that structure to help it partly dissolve or become metabolized over time. Bandage or suture 1200 may be a removable surface applique having a flat shape and an area, or it may consist of a one dimensional implantable structure such as a thread to hold lacerated or cut tissue together in abutment to confer healing of the torn or cut surfaces. In particular, the material of the surface applique may be a nonwoven cotton matrix having a surface coated with gelatin containing anisotropic polyhydroxylated fullerenes 1210, 1220, 1230, where that surface is then heated and dried to form an adherent oxidized gelatin coating. The material of suture thread may be any of those preferably non-braided forms that are commercially optimized for tensile strength to include, without limitation, poliglecaprone suture material (Monocryl® suture material available from Ethicon, Inc., Somerville, N.J., USA), or polypropylene suture material (Prolene® suture material also available from Ethicon, Inc.). The anisotropic polyhydroxylated fullerenes 1210, 1220, 1230 can be stabilized in beta-cyclodextrine, and the stabilized anisotropic polyhydroxylated fullerenes 1210, 1220, 1230 then can be sprayed onto the polymer mesh by a conventional oxygen plasma treatment. The depicted bandage or suture 1200 can be infused with, and contains, fullerenes being a dispersion of fullerol-8 indicated by 1210, 1220, or a mixture of polyhydroxylated fullerenes 1230 where the fullerol-x can be primarily x=8 (fullerol-8), or other fullerenes of few covalent substituents being of low functionalization (x=less than 22) capable of conferring antimicrobial properties. These fullerenes can fight epidermal bacterial wound infections and bacteria adherent to subdermal suture thread materials that are resistant to conventional antibiotics, especially when these materials are enhanced by the use of anisotropic functionalized polyhydroxylated fullerene. Antimicrobial action also can be obtained by exposure of the bandage or suture 1200 to light 1260 being sunlight, or light 1260 being the irradiation used in photodynamic therapy typically of red light between about 730 nm to about 760 nm, or infrared light from about 760 nm to about 860 nm.

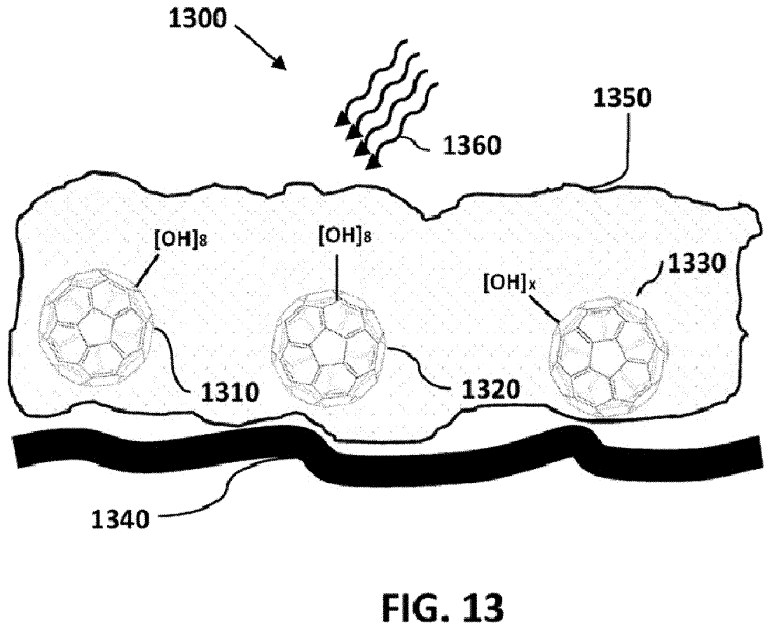

FIG. 13 depicts topical ointment 1350 used to promote healing of the skin or other surface tissue such as the externally exposed tissues of the eye, where the ointment contains a dispersion of fullerol-8 indicated by 1310, 1320, or a mixture of polyhydroxylated fullerenes 1330 where the fullerol-x can be primarily x=8 (fullerol-8), or other fullerenes of few covalent substituents being of low functionalization (x=less than 22) capable of conferring antimicrobial properties. The depicted fullerenes can fight bacterial infections resistant to conventional antibiotics, especially when these are enhanced by both the use of anisotropic functionalized polyhydroxylated fullerenes having exposure of the salve or ointment to light 1360 being sunlight, or light 1360 being the irradiation used in photodynamic therapy typically of red light from about 730 nm to about 760nm, or infrared light from about 760 nm to about 860 nm.

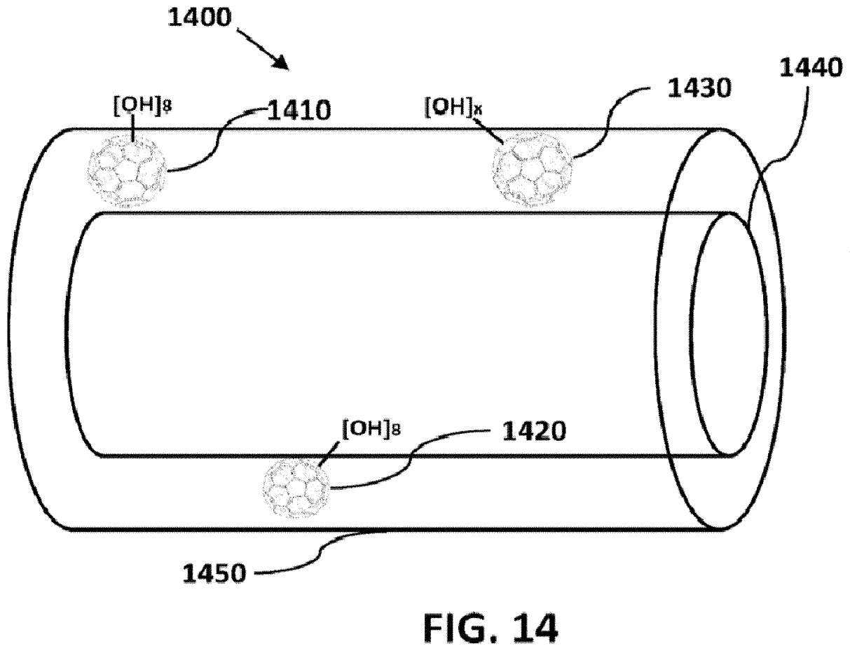

FIG. 14 depicts a tubular stent 1400, which is a support placed temporarily inside a blood vessel, canal, or duct to aid healing or relieve an obstruction, or a support surgically placed at the node of a meridian in the human body for the purpose of providing acupuncture by means of a sterilized needle inserted into the orifice 1440 of this tubular stent 1400. Tubular stent material 1450 can be infused with fullerol-8 indicated by 1410, 1420, or with a mixture of polyhydroxylated fullerenes 1430 where the fullerol-x can be primarily x=8 (fullerol-8), or other fullerene of few covalent substituents being of low functionalization (x=less than 22). Deeply implanted stent material 1450 may not be directly capable of illumination by light at the site of implantation, however the perfusion of blood though the adjacent tissues and also into the material of 1450 will contain red blood cells that contain dissolved oxygen as well as singlet oxygen which are capable of activating the polyhydroxylated fullerenes 1410, 1420, 1430 to confer localized antibiotic and antimicrobial properties. The material of the stent may be, for example and without limitation, a commercially available cross-linked collagen-containing tissue scaffold, or a sewn goat intestine processed to remove genetic material and proteins capable of initiating an immune response, or a vein harvested from a different area of the body and treated with fullerenes by ultrasonic agitation in a blood plasma containing concentrated polyhydroxylated fullerenes capable of penetrating that tissue prior to implantation back into a different part of the same host to confer protective antibiotic and anti-coagulant properties to the material of the stent. One form of the stent may be, for example, a commercially available polypropylene mesh used for hernia repair. Sometimes the shape of the stent material 1450 is not always tubular, as body cavities or soft tissues requiring such scaffolds have natural cavities and shapes that are dictated by biological function, therefore requiring the form of an impression or cast of a part or body cavity, used to maintain pressure so as to promote healing, especially of a skin graft. Thus, stent 1400 also represents a tubular outside section of an arm or a leg, where the exterior skin has been burned away and now requires the application of a stent in the form of a sleeve 1450 applied from the outside of the remaining tissues to promote skin healing. Furthermore, stent 1400 can be representative of, without limitation, an acupuncture support, or medical wound drain material or wound packing material.

Variations, combinations, and modifications may be made in the constituents of baked goods, candies, and beverages to which various amounts of food grade poly-hydroxylated fullerenes, such as fullerol-8, may be formulated and processed with food-grade additives and components. Thus, a medicinal, nutraceutical, or food fullerene composition is provided herein. Selected embodiments are directed to a charge networked fullerol that can be stabilized in an ionomer nano-dispersion composition for human consumption with the intent to confer long term prophylactic antioxidant, antiviral, and protective neurological protection. An edible and non-toxic ionomeric matrix may be made, which is a mixture of hydrogen bonded and conjugated molecules having melting points equal to or below about 100° C. Many of the ingredients used to make edible ionomeric compositions are functionally present in natural honey. These ingredients, with optional proline and beta-cyclodextrin addition, can be able to form long term stable nano-dispersions of fullerenes by providing intermolecular induced charge networks and screened molecular nanoparticle stabilizations which avoid nanoparticle sedimentation or agglomeration at high concentrations found in other types of dispersion media. This edible, hydrogen bonded, conjugated charge-induced ionomer matrix is herein termed ‘fullerol-8 ionomer’.

In embodiments, the fullerol-8 ionomer desirably contains more than 1000 parts per million of optional proline and flavinols, and desirably about 1 percent by weight of beta-cyclodextrin. This ionomer is capable of forming additional conjugates with certain types of organic food molecules, especially proline, creatine, or a wide variety of proteins, lipids, and carboxylic acids capable of stabilizing the fullerol-8 in liquid carrier media containing water. This ionomeric network is resistant to sedimentation of polyhydroxylated fullerenes.

The fullerol-8 ionomer becomes diluted and digested over time, however the charge screening effects of the ionomer in charge-networked association with fullerenes are especially difficult to disrupt because of a shear induced electronic charging of the fullerol-8 molecules having formed complexes with large soluble organic molecules. Even under dilute conditions in other media, the shear-induced charge network stabilization of fullerol-8 ionomer avoids the formation of a sedimentation process where agglomerates and precipitates grow in size, as would be the case in unprotected self-cohesion induced by the nano-particle polarization of bare uncharged fullerene attraction to like bare uncharged fullerene molecules.

The ionomer mixture may include the composition of nominally 1% protein containing nitrogen capable of van-der-Waals attraction to the fullerenes, and limits or avoids undesirable agglomeration of highly concentrated fullerenes in the dispersion process.

Many of the ionomer constituents used to form charge stabilized conjugates are naturally present in honey. Increasing the amount of inorganic ionomeric salts, for example, by the addition of inorganic sodium or potassium, enables the creation of an edible ionomer capable of charge coupling to negatively charged fullerene molecules at high concentrations after sufficient shear-induced charging for long term stability of the ionomeric charged network.

The somatic dilution of a charge-stabilized ionomeric fullerol molecular suspension, to include those that are metabolically stabilized in human blood plasma, may have strong antiviral properties arising from fullerene opening of viral capsid coatings by exploding the virus particles. These moieties are attracted in dilute form along with some of the charge- stabilized ionomer shell molecules to locations stored within the lipid bilayer of living cells. Virus particle are also attracted to such lipids as a characteristic property enabling viral cellular invasion.

In embodiments, a food is created by blending a predetermined amount of a polyhydroxylated fullerene with an acceptable adjuvant, an accepted carrier, or both. In one exemplary food using fullerol-8, a candy may be made with about 200 parts per million fullerol-8, about 3% by wt. sugar (as sucrose), about 1% by wt. proline, about 2% by wt. magnesium citrate, about 2% by wt. citric acid, about 4 ounces of gelatin, and 2-3 oz. packages (or one 6 oz. package) of a commercial flavored gelatin dessert, prepared with the foregoing adjuvants or carriers. FD&C food coloring may be included in the candy. When mixed and poured into a mold and allowed to set, a gummy-type candy can be produced. The presence of at least 200 ppm of fullerol-8 can be therapeutic for basic anti-oxidant purposes, although there is no recommended daily allowance of fullerol-8, or any known limitation for this substance in food at this time. Approximately 200 ppm concentration of fullerol-8 can be prepared from direct volume dilution of 1500 parts per million of fullerol-8 stock solution as described in FIG. 7, by diluting 133.4 ml of this aqueous or water based solution with sufficient pure water to achieve a volume of 1000 ml for the purpose of a food supplement and to act as a food preservative. The purpose of proline is to stabilize the fullerol-8 nanoparticles as a dispersion and to prevent the agglomeration of fullerol-8 into clusters greater than about 100 nanometers. The purpose of magnesium citrate is to provide magnesium mineral supplement at a concentration less than the recommended daily allowance of elemental magnesium. The purpose of the citrate ion is to act as a food preservative. The purpose of the cross-linking of the polypeptides is to preserve the less than 200 nanometer agglomerate size of polyhydroxylated fullerenes by steric confinement into the polymer chain network. Both unflavored gelatin and commercially available Jell-O® brand gelatin mixture can be provided in the same mixture to create a candy of sufficient hardness to provide a hard chew food experience.

In another exemplary food medicament using fullerol-8, a fortified beverage may be made with water, about 200 parts per million fullerol-8, about 3% by wt. of sugar (as sucrose), about 1% by wt. of proline, about 2% by wt. of magnesium citrate, and about 2% by wt. of citric acid. FD&C food coloring may be included in the beverage. Approximately 200 ppm concentration of fullerol-8 can be prepared from direct volume dilution of about 1500 parts per million of fullerol-8 stock solution of FIG. 7, by diluting about 133.4 ml of this aqueous or water based solution with sufficient pure water to achieve a volume of about 1 liter. This beverage may be administered in two separate 500 ml serving containers, where one such container constitutes one serving having 750 ppm of fullerol-8 per serving. However, it may be desirable to maintain the concentration of fullerol-8 at 1500 ppm, and to administer two servings of 100 ml for the purpose of portability and ease of pocket transport of this serving size and concentration. There is no known or recommended limit or daily allowance of fullerol-8 to define or limit the number of such servings to drink as a beverage.

In another exemplary food medicament using the food grade polyhydroxylated fullerenes of the present invention, especially that of the preferred fullerol-8, a concentrated fullerol oil of substantial purity may be supplied as a raw material meeting USP food grade requirements, being created by vacuum drying of the water solution of FIG. 7, while taking care not to destroy the polyhydroxylated fullerenes by excessive heating, by keeping the vacuum process temperature below 60 degrees C. Optionally, 0.02% by weight of proline can be added to stabilize aggregates of this concentrated fullerol-8. Substantially pure (99.98%) fullerol-8 may be used as an additive to create food or beverage products, or to create skin care products. There is no known or recommended limit or daily allowance of fullerol-8 at present that may be used to define or limit the concentration of fullerol-8, however some concentrations or serving size may be selected to achieve the final product design purpose.

In still another exemplar food using fullerol-8, a candy may be made using 200 ml of 1500 ppm of fullerol-8 diluted into 454 grams or about 16 ounces of confectionary chocolate, for example, 60% cacao, in which the solution is brought to a simmer for about 60 minutes to remove much of the water and to ensure this mixture is mixed thoroughly before pouring into molds to create one piece that is a solid serving having some convenient or decorative shape, but conveniently being about 23 grams to make about 20 servings. The density of commercially-purchased chocolate mixture varies depending on ratio of milk, cocoa, and other ingredients, but is typically close to about 1.325 grams per cubic centimeter. One piece of such chocolate will contain about 44 ppm of fullerol-8 and may constitute one serving. There is no known or recommended limit or daily allowance of either chocolate, or fullerol-8, to define or limit the number of such servings to be eaten. Although the foregoing discussions of FIGS. 9 and 10 are focused on the polyhydroxylated fullerene, fullerol-8, other species of polyhydroxylated fullerene may be similarly prepared and used, as described in the foregoing discussion of FIG. 11.

The purpose of having an unstable dispersion of two edible or non-toxic immiscible liquids (e.g. oil and water) is to create high surface areas between unlike liquid media, wherein the surface free energy at the interface of the two liquids is reduced. This is accomplished by shearing at high rates to create many bubbles or “vesicles” of the minority water-like liquid in the majority oily-like liquid. The important result is the creation of large liquid-liquid interfacial surface area. When nanoparticles locate into this interface or “meniscus”, the chemical reactivity at that location becomes entropically favored. Theoretically, the exposure of different solvent environments induces an alteration of the electronic density of states (DOS) in the carbon atoms composing the C60 cage. The DOS is the probability of finding an electron in a resonance bond of the fullerene. This probability undergoes a transition at the atoms of carbon located along the circumference of the spherical fullerene cage at the meniscus where it is suspended between polar and non-polar solvent at either liquid side, thereby increasing their reactivity at this meniscus location. Also, reactive oxygen species and free radicals such as H (dot or ⋅) or OH-(dot or ⋅) tend to migrate into this region, because unlike substances migrate into and collect at interfaces as a result of entropy state increase and thus energy state decrease. The combination of reduced surface free energy and highly-reactive radicals at the same place, increases the likelihood or probability to allow the hydroxylation reaction to proceed at the fullerene circumference along the liquid-liquid interface, with reduced energy and thus reasonably fast reaction rate, wherein such probability of reactivity it will not be favored when the fullerene cage is surrounded by pure oil or suspended in pure water. The lack of a non-toxic synthesis that is free of phase transfer catalysts and industrial solvents has posed a barrier to routine non-toxic and facile production of polyhydroxylated fullerenes having few numbers of hydroxylations. It is likely that the role of entropy to allow this reaction to progress with significant yield has been greatly underappreciated.

There is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations or incremental alterations to the drawings, steps, methods, or processes, depicted therein without departing from the spirit of the invention. All of these variations are considered to be within the scope of the present invention. Hence, specific structural and functional details disclosed in relation to the exemplary embodiments described herein are not to be interpreted as limiting, but merely as a representative basis for teaching one skilled in the art to variously employ the present invention in virtually any appropriate form, and it will be apparent to those skilled in the art that the present invention may be practiced without these specific details.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but defined in accordance with the foregoing claims appended hereto and their equivalents.

What is claimed is:

1. A method for synthesizing a medicinal, nutraceutical, or food fullerene composition, comprising:
providing anisotropy in polar and non-polar C60 fullerene hemispheres to create one face of C60 fullerene having a small number of OH-groups clustered to the polar face;
providing a predetermined amount of a polyhydroxylated fullerene from C60 fullerene; and
blending the predetermined amount of the polyhydroxylated fullerene with an acceptable ionomer or an acceptable carrier or both.

2. The method of claim 1, wherein the polyhydroxylated fullerene includes fulleroDx'and the predetermined amount comprises about 200 ppm, wherein ‘x’ is less than 22.

3. The method of claim 1, wherein the polyhydroxylated fullerene includes fullerol-‘x’ and the predetermined amount comprises about 500 ppm, wherein ‘x’ is less than 22.

4. The method of claim 3, wherein the acceptable carrier comprises about 16 ounces of chocolate.

5. The method of claim 2, wherein the acceptable ionomer comprises a mixture of about 3% by wt. of sucrose, about 1% by wt. of proline, about 0.2% by wt. of magnesium citrate, and about 1% by wt. of beta-cyclodextrin.

6. The method of claim 2, wherein the acceptable ionomer comprises honey.

7. The method of claim 5, wherein the acceptable carrier comprises water.

8. The method of claim 5, wherein the acceptable carrier comprises a gelatin.

9. A medicinal, nutraceutical, or food fullerene composition, comprising:
a predetermined amount of anisotropic functionalized polyhydroxylated fullerene; and an acceptable ionomer or an acceptable carrier or both.

10. The fullerene composition of claim 9, wherein the anisotropic functionalized polyhydroxylated fullerene includes fullerol-8, and the predetermined amount of fullerol-8 comprises about 200 ppm.

11. The fullerene composition of claim 9, wherein the anisotropic functionalized polyhydroxylated fullerene includes fullerol-8 and wherein the acceptable carrier comprises about 16 ounces of chocolate.

12. The fullerene composition of claim 10 wherein the acceptable ionomer comprises a mixture of about 3% by wt. of sucrose, about 1% by wt. of proline, about 0.2% by wt. of magnesium citrate, and about 1% by wt. of beta-cyclodextrin.

13. The fullerene composition of claim 12, wherein the acceptable carrier comprises water.

14. The fullerene composition of claim 12, wherein the acceptable carrier comprises a gelatin.

15. The fullerene composition of claim 10, wherein the acceptable ionomer comprises bee honey.

16. The fullerene composition of claim 10, wherein the acceptable carrier comprises about 5% by volume of ethanol.

17. The fullerene composition of claim 9, wherein the anisotropic functionalized polyhydroxylated fullerene comprises 99.98% fullerol-8.

18. The fullerene composition of claim 9, wherein the anisotropic functionalized polyhydroxylated fullerene comprises fullerol-8 stabilized with beta-cyclodextrin and wherein the acceptable carrier comprises human blood plasma.

19. A method of killing or inhibiting the growth of a bacteria or a virus, comprising contacting the bacteria or the virus with an effective antibacterial or antiviral amount of the fullerene composition of claim 9.

20. The method of claim 19, where a substrate impregnated with the composition of claim 9 comprises a medical bandage material, a medical packing material, a medical drain material, a suture thread, a stent, or a topical ointment.

21. A method of augmenting the mental acuity of a human comprising administering an effective amount of the fullerene composition of claim 9.

22. The method of claim 19, further comprising administering an effective amount of transcranial direct current stimulation to a human brain.

23. A method of treating or managing a toxic chemical ingestion by a mammal, comprising administering an effective amount of the fullerene composition of claim 9.

24. The method of claim 22 further comprising administering a photodynamic therapy.
